(19) United States
(12) Patent Application Publication
(10) Pub. No.: US 2024/0117897 A1
(43) Pub. Date: Apr. 11, 2024

(54) CONDUIT ATTACHMENT DEVICE

(71) Applicant: Joseph D. Minder, Charlotte, NC (US)

(72) Inventor: Joseph D. Minder, Charlotte, NC (US)

(21) Appl. No.: 18/483,822

(22) Filed: Oct. 10, 2023

Related U.S. Application Data
(60) Provisional application No. 63/379,007, filed on Oct. 11, 2022.

(51) Int. Cl. F16L 3/227; F16L 3/10

(52) U.S. Cl. CPC F16L 3/227; F16L 3/1041

(57) ABSTRACT

A conduit attachment device may include a retainer having a retainer base, a first retainer arm, a second retainer arm, a retainer strap, and a fastener. The first retainer arm and the second retainer arm may be coupled to the retainer base to form a retainer channel. The retainer strap may be coupled to the first retainer arm and the fastener may be coupled to the second retainer arm. A holder may be coupled to the retainer, and the holder may have a holder base, a first holder arm, and a second holder arm. The first holder arm and the second holder arm may be coupled to opposing ends of the holder base to form a holder channel. A first clip may be coupled to the first holder arm, and a first clip channel may be formed between a first holder arm, a first clip base, and a first clip arm of the first clip.

### CROSS REFERENCE TO RELATED APPLICATIONS

This application claims priority to and the benefit of the filing date of U.S. Provisional Application No. 63/379,007, filed on Oct. 11, 2022, entitled “Bedrail Attachment Device for Routing Lines”, which is hereby incorporated by reference in its entirety.

### FIELD OF THE INVENTION

This patent specification relates to the field of medical care equipment. More specifically, this patent specification relates to a device for removably coupling lines, wires, and conduits to a bed rail of a patient bed and other medical care equipment.

### BACKGROUND

While the inventor was working as a RN in the ICU environment, he observed the increasing need for an easy to use tube management system. Considering the Critical Care environment, and how sick patients are, there are an ever-increasing number of IV lines, tubes, device wires etc. IV lines can infuse any number of medicines ranging from antibiotics to life saving blood pressure medicines. It's not uncommon to have 6 or more IV lines infusing on an ICU patient at once. Until now, nurses have used various means to organize these lines, including taping medicine cups and test tubes to bed rails to serve as an anchoring device. Subsequently, the inventor identified a need for a novel attachment device for routing lines to solve this tube management problem. Currently, there are no devices that we have found that quickly attach to a hospital bed rail and provide this utility. Some hospital beds have built in articulating arms that have the ability to aid in wire consolidation, but are typically inconveniently located at the head of the bed—creating a less than ergo dynamic organizational system—as most IV tubing is running from IV pumps located on the sides of the bed. Additionally, these features are not included on all beds, so this functionality is limited on a bed-by-bed basis.

Therefore, a need exists for a novel device for removably coupling lines, wires, and conduits to a bed rail and other medical care equipment. A further need exists for a novel conduit attachment device that does not suffer from the above mentioned drawbacks.

### BRIEF SUMMARY OF THE INVENTION

A conduit attachment device which may be used for removably coupling lines, wires, and conduits to a bed rail of a patient bed and other medical care equipment is provided. In some embodiments, the device may include a retainer having a retainer base, a first retainer arm, a second retainer arm, a retainer strap, and a fastener. The first retainer arm and the second retainer arm may be coupled to the retainer base, and a retainer channel may be formed between the first retainer arm, the retainer base, and the second retainer arm. The retainer strap may be coupled to the first retainer arm and the fastener may be coupled to the second retainer arm. The fastener may be configured to be coupled to the retainer strap. A holder may be coupled to the retainer, and the holder may have a holder base, a first holder arm, and a second holder arm. The first holder arm and the second holder arm may be coupled to the holder base, and a holder channel may be formed between the first holder arm, the holder base, and the second holder arm. A first clip may be coupled to the holder, and the first clip may have a first clip base, and a first clip arm. The first clip base may be coupled to the first holder arm, and a first clip channel may be formed between the first holder arm, the first clip base, and the first clip arm. Generally, a portion of an object, such as a bedrail of a hospital bed, may be positioned in the holder channel, and the device may be coupled to the object by positioning the retainer strap around the object and coupling the fastener to the retainer strap. One or more lines, wires, conduits, etc., may then be positioned in the holder channel and/or the first clip channel so that the lines, wires, conduits etc., may be supported by the device to allow for the clinician to easily identify and consolidate all of their lines in a central location.

Numerous objects, features and advantages of the present invention will be readily apparent to those of ordinary skill in the art. Some example objects of the present invention are listed below.

One object of the present invention is to provide a conduit attachment device that can give value to bedside clinicians in Organization & Efficiency: this device allows for the clinician to easily consolidate all of their lines into a central location; the one or more clips which may be used as primary IV clip(s) allows a RN the ability to quickly identify and access their KVO line to push meds for bedside procedures like a tracheostomy, bronchoscopy etc. (KVO=keep vein open); an IV line that typically has maintenance IV fluids like normal saline or lactated ringers going in at a slow rate and is used as the primary line for pushing emergency procedural meds like sedatives, paralytics and narcotics. It is an essential line to use and keep separate and distinguishable from other lines; therefore, the one or more clips which may be used as primary IV clip(s) of the device has the added value of this safe and efficient organizational utility.

Another object of the present invention is to provide a conduit attachment device that can give value to bedside clinicians in Safety: With so many lines and wires, it is not uncommon for them to get lodged in between a bed rail and a mattress: if a patient rolls or is combative, the tension from a misplaced line can pull out of a patient—causing bleeding and potentially life threatening issues (i.e. dislodged arterial line). Having the one or more clips which may be used as primary IV clip(s) of the device increases patient safety since the nurse can clearly see which line is dedicated for specific medications and will prevent medication errors.

Another object of the present invention is to provide a conduit attachment device that can give value to bedside clinicians in Cost Savings: The disclosed device for routing lines can aid in cost reduction by reducing medical error and prevent costly mistakes. It will streamline the nurse's workflow thereby allowing more time to devote to patient care.

### BRIEF DESCRIPTION OF THE DRAWINGS

Some embodiments of the present invention are illustrated as an example and are not limited by the figures of the accompanying drawings, in which like references may indicate similar elements and in which:

FIG. 1—FIG. 1 depicts a perspective view of an example of a conduit attachment device according to various embodiments described herein.

FIG. 2—FIG. 2 illustrates another perspective view of an example of a conduit attachment device according to various embodiments described herein.

FIG. 3—FIG. 3 shows an elevation view of an example of a conduit attachment device according to various embodiments described herein.

FIG. 4—FIG. 4 depicts a perspective view of an example of a retainer according to various embodiments described herein.

FIG. 5—FIG. 5 illustrates an elevation view of an example of a holder according to various embodiments described herein.

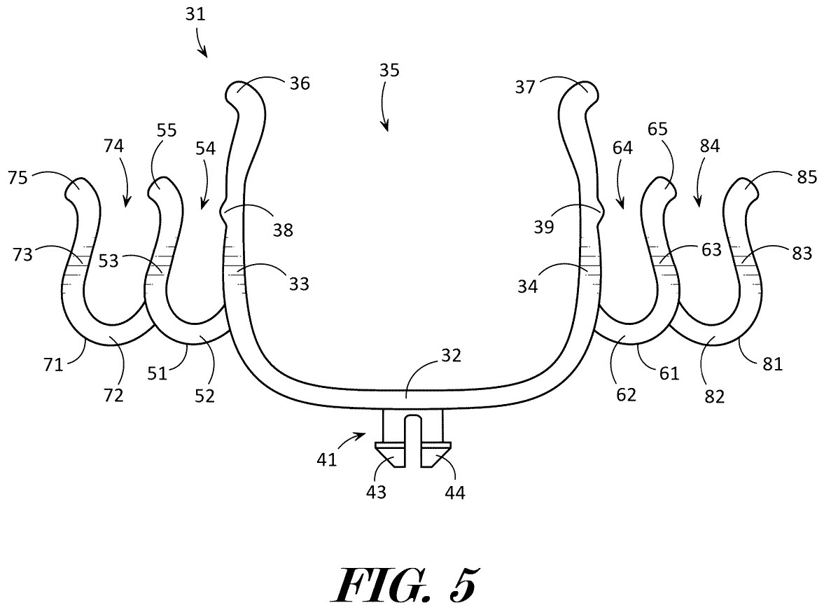

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

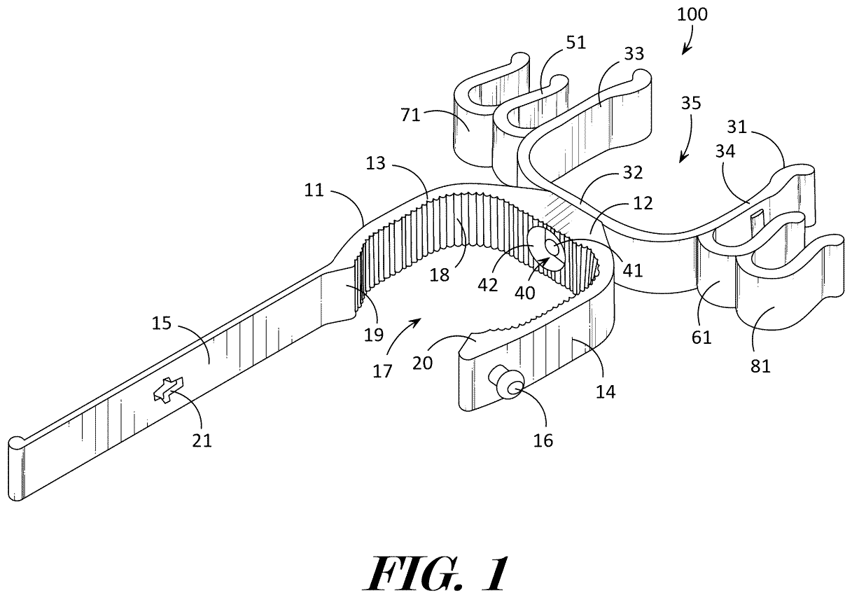

For purposes of description herein, the terms “upper,” “lower,” “left,” “right,” “rear,” “front,” “side,” “vertical,” “horizontal,” and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first,” “second,” etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 20% of the specified number. Additionally, as used in this application, the term “substantially” means that the actual value is within about 10% of the actual desired value, more preferably within about 5% of the actual desired value and even more preferably within about 1% of the actual desired value of any variable, element or limit set forth herein.

A new conduit attachment device is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

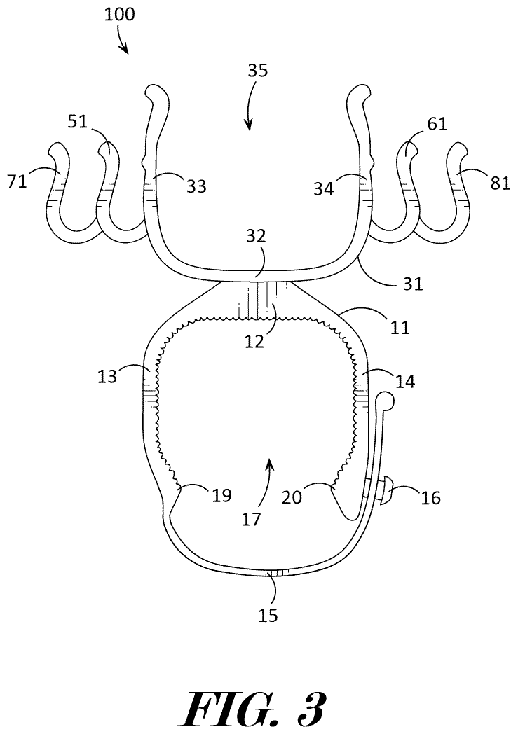

The present invention will now be described by example and through referencing the appended figures representing preferred and alternative embodiments. FIGS. 1-3 illustrate an example of a conduit attachment device (“the device”) 100 according to various embodiments. In some embodiments, the device 100 may comprise a retainer 11 having a retainer base 12, a first retainer arm 13, a second retainer arm 14, a retainer strap 15, and a fastener 16. The first retainer arm 13 and the second retainer arm 14 may be coupled to the retainer base 12 and separated from each other so that a retainer channel 17 is formed between the first retainer arm 13, the retainer base 12, and the second retainer arm 14. The retainer strap 15 may be coupled to the first retainer arm 13 and the fastener 16 may be coupled to the second retainer arm 14. The fastener 16 may be configured to be coupled to the retainer strap 15. A holder 31 may be coupled to the retainer 11, and the holder 31 may have a holder base 32, a first holder arm 33, and a second holder arm 34. The first holder arm 33 and the second holder arm 34 may be coupled to opposing ends of the holder base 32, and a holder channel 35 may be formed between the first holder arm 33, the holder base 32, and the second holder arm 34. A first clip 51 may be coupled to the holder 31, and the first clip 51 may have a first clip base 52, and a first clip arm 53. The first clip base 52 may be coupled to the first holder arm 33, and a first clip channel 54 may be formed between the first holder arm 33, the first clip base 52, and the first clip arm 53. Generally, a portion of an object, such as a bedrail of a hospital bed, may be positioned in the holder channel 35, and the device 100 may be coupled to the object by positioning the retainer strap 15 around the object and coupling the fastener 16 to the retainer strap 15. One or more lines, wires, and conduits may then be positioned in the holder channel 35 and/or the first clip channel 54 so that the lines, wires, and conduits may be supported by the device 100 to allow for the clinician to easily identify and consolidate all of their lines in a central location.

The device 100 may comprise a retainer 11 which may be configured to couple, and more preferably to removably, couple the device 100 to a plurality of different sized and shaped objects, such as a bedrail of a hospital bed. In some embodiments, a retainer 11 may include a first retainer arm 13 and a second retainer arm 14 that may be coupled to a retainer base 12. The first retainer arm 13 and second retainer arm 14 may be separated from each other so that a retainer channel 17 is formed between the first retainer arm 13, the retainer base 12, and the second retainer arm 14. Preferably, an object, such as a bedrail of a hospital bed, may be placed in the retainer channel 17 so that opposing portions of the object may be contacted and gripped by the two retainer arms 13, 14. Optionally, the retainer 11 may comprise three or more retainer arms 13, 14, and one or more retainer channels 17 may be formed between two or more retainer arms 13, 14, and the retainer base 12.

The retainer base 12, first retainer arm 13, and second retainer arm 14 may be configured in any size and shape to form a retainer channel 17 of any size and shape. In some embodiments, a first retainer arm 13 and a second retainer arm 14 may be coupled to opposite sides or opposite ends to the retainer base 12 so that a relatively longer retainer base 12 may be used to form a relatively wider retainer channel 17. In further embodiments, a first retainer arm 13 and a second retainer arm 14 may be generally elongated in shape and may extend away from the retainer base 12 so that relatively longer retainer arms 13, 14, may be used to form a relatively deeper retainer channel 17. In preferred embodiments, the first retainer arm 13 may be elongated in shape and may extend away from the retainer base 12 in a direction that is substantially parallel (plus or minus degrees) to a direction that the second retainer arm 14, also being elongated in shape, extends away from the retainer base 12.

In preferred embodiments, the retainer base 12 and/or one or more retainer arms 13, 14, may be made from or may comprise a resilient material that may be flexible to allow slight deformation and resilient so as to return to its original shape after deformation. In preferred embodiments, a resilient material may comprise a natural and/or synthetic rubber material, such as latex rubber, silicone foam, silicone rubber, rubber foam, urethane foam, plastic foam, neoprene foam, latex foam rubber, polyurethane foam rubber, forms of the organic compound isoprene, Polyacrylate Rubber, Ethylene-acrylate Rubber, Polyester Urethane, flexible plastics, such as high-density polyethylene (HDPE), polyvinyl chloride (PVC), polypropylene (PP), Polystyrene (PS), Polycarbonate (PC), low density polyethylene (LDPE), or any other flexible material including combinations of materials.

In some embodiments, the retainer base 12 and/or one or more retainer arms 13, 14, may comprise texturing 18. Preferably texturing 18 may be disposed on all or portions of the retainer base 12 and/or one or more retainer arms 13, 14, that form the retainer channel 17. Texturing 18 may comprise a plurality of ridges, bumps, peaks and valleys, or any other texturing or surface treatment which may increase the frictional resistance of the retainer base 12 and/or one or more retainer arms 13, 14, from moving relative to an object that is in the retainer channel 17 and that is contacting the texturing 18. Optionally, the retainer base 12 and/or one or more retainer arms 13, 14, may comprise a non-slip material, such as rubber, silicone, or other friction enhancing material, which may be disposed on all or portions of the retainer base 12 and/or one or more retainer arms 13, 14, that form the retainer channel 17.

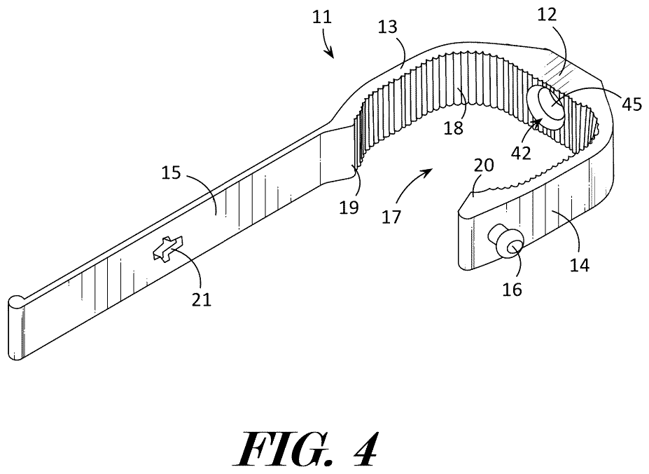

In some embodiments, the first retainer arm 13 may comprise a first retainer barb 19 and/or the second retainer arm 14 may comprise a second retainer barb 20. Generally, a retainer barb 19, 20, may comprise a ridge, bump, or other type of projection which may extend away from a retainer arm 13, 14, and extend into or towards the retainer channel 17. Preferably, a retainer barb 19, 20, may comprise a projection near the free end of a retainer arm 13, 14, which may increase the frictional resistance of the retainer arm 13, 14, against an object in the retainer channel 17 to help prevent the object from being pulled away from the retainer base 12 and out of the retainer channel 17. In some embodiments, a retainer barb 19, 20, may comprise a triangular prism shaped projection, as perhaps best shown in FIGS. 1-4, although a retainer barb 19, 20, may be configured in any shape and size. In preferred embodiments, the first retainer arm 13 may comprise a first retainer barb 19 and the second retainer arm 14 may comprise a second retainer barb 20 and the retainer barbs 19, 20, may face towards or extend towards each other.

In some embodiments, the device 100 may comprise a retainer strap 15 which may be configured to wrap around a portion of an object that is received in the retainer channel 17. In preferred embodiments, a retainer strap 15 may be coupled to the first retainer arm 13 and the retainer strap 15 may be coupled, and more preferably removably coupled, to a fastener 16 that may be coupled to the second retainer arm 14. Generally, a retainer strap 15 may comprise an elongated length of flexible material, such as flexible plastic, nylon webbing, etc. In some embodiments, a retainer strap 15 may be coupled to a fastener 16 and configured to tension the retainer arms 13, 14, towards each other and into contact with a portion of an object that is received in the retainer channel 17. In further embodiments, a retainer strap 15 may be configured to wrap around and contact a portion of an object that is received in the retainer channel 17.

A retainer strap 15 and a fastener 16 may comprise any suitable device or method for coupling, and more preferably for removably coupling, two objects together. In some embodiments, a retainer strap 15 may have a fastener aperture 21, and a fastener 16 may be configured to be coupled to the retainer strap 15 by press-fitting and inserting the fastener 16 into the fastener aperture 21. In further embodiments, a retainer strap 15 and a fastener 16 may comprise a magnetic material which may be used to magnetically couple the retainer strap 15 and fastener 16 together. In still further embodiments, a retainer strap 15 and a fastener 16 may comprise a hook-and-loop fastener, such as Velcro.

The device 100 may comprise a holder 31 which may be configured to hold, support, or otherwise position one or more lines, wires, and conduits. In some embodiments, a holder 31 may include a first holder arm 33 and a second holder arm 34 that may be coupled to a holder base 32. The first holder arm 33 and second holder arm 34 may be separated from each other so that a holder channel 35 is formed between the first holder arm 33, the holder base 32, and the second holder arm 34. Preferably, a portion of one or more lines, wires, and conduits may be placed in the holder channel 35 and the holder arms 33, 34, may keep the portion of the one or more lines, wires, and conduits in the holder channel 35. Optionally, the holder 31 may comprise three or more holder arms 33, 34, and one or more holder channels 35 may be formed between two or more holder arms 33, 34, and the holder base 32.

The first holder arm 33, the holder base 32, and the second holder arm 34 may be configured in any size and shape to form a holder channel 35 of any size and shape. In some embodiments, a first holder arm 33 and a second holder arm 34 may be coupled to opposite sides or opposite ends to the holder base 32 so that a relatively longer holder base 32 may be used to form a relatively wider holder channel 35. In further embodiments, a first holder arm 33 and a second holder arm 34 may be generally elongated in shape and may extend away from the holder base 32 so that relatively longer holder arms 33, 34, may be used to form a relatively deeper holder channel 35. In preferred embodiments, the first holder arm 33 may be elongated in shape and may extend away from the holder base 32 in a direction that is substantially parallel (plus or minus degrees) to a direction that the second holder arm 34, also being elongated in shape, extends away from the holder base 32.

In preferred embodiments, a first holder arm 33 may comprise a first holder distal end 36 and a second holder arm 34 may comprise a second holder distal end 37, and the first holder distal end 36 and the second holder distal end 37 may be flared away from each other. Holder distal ends 36, 37, may comprise the end of the holder arms 33, 34, that are farthest from the holder base 32. Generally, holder distal ends 36, 37, that are flared away from each other may facilitate the entry of lines, wires, and conduits into the holder channel 35.

In preferred embodiments, the holder base 32 and/or one or more holder arms 33, 34, may be made from or may comprise a resilient material that may be flexible to allow slight deformation and resilient so as to return to its original shape after deformation. In preferred embodiments, a resilient material may comprise a natural and/or synthetic rubber material, such as latex rubber, silicone foam, silicone rubber, rubber foam, urethane foam, plastic foam, neoprene foam, latex foam rubber, polyurethane foam rubber, forms of the organic compound isoprene, Polyacrylate Rubber, Ethylene-acrylate Rubber, Polyester Urethane, flexible plastics, such as high-density polyethylene (HDPE), polyvinyl chloride (PVC), polypropylene (PP), Polystyrene (PS), Polycarbonate (PC), low density polyethylene (LDPE), or any other flexible material including combinations of materials.

A retainer 11 and a holder 31 may be coupled together with any suitable coupling method. In some embodiments, a retainer base 12 of a retainer 11 may be coupled to the holder base 32 of a holder 31. Optionally, a retainer 11 and a holder 31 may be coupled together by being integrally formed or molded together.

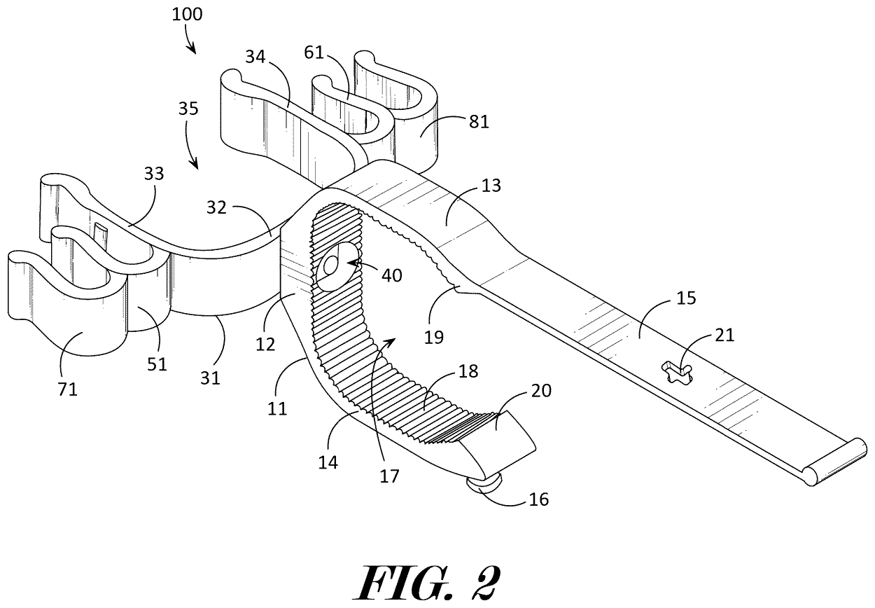

In preferred embodiments, a retainer 11 and a holder 31 may be movably coupled together so that the orientation of the retainer arms 13, 14, relative to the holder arms 33, 34, may be changed or repositioned as perhaps best shown by FIGS. 1 and 2. In further preferred embodiments, a holder 31 and a retainer 11 may be rotatably coupled together via a movable coupling 40 so that the holder 31 and retainer 11 may rotated or pivoted relative to each other as shown in the change in orientation of the retainer 11 relative to the holder 31 in FIGS. 1 and 2.

In some embodiments, a movable coupling 40 may comprise a male member 41 and a female member 42, and the male member 41 may be rotatably coupled within the female member 42 so that the male member 41 and female member 42 may enable the holder 31 and retainer 11 to be rotated or pivoted relative to each other. For example, a male member 41 may comprise two barbed male arms 43, 44, that are able to resiliently flex towards each other and that may be press fit into a female member 42 that may comprise a cylindrical opening 45. Once pressed into the opening 45 the barbed male arms 43, 44, may flex against the opening 45 and be prevented from being withdrawn from the opening 45 while allowing the arms male arms 43, 44, to rotate within the opening 45. As another example, a male member 41 and a female member 42 may be configured as a ball joint so that the male member 41 may comprise a bearing stud that is movably and rotatably coupled within a female member 42 that comprises a socket. As another example, a male member 41 may comprise a bolt, rivet, or other fastener that may be rotatably coupled within a female member 42 that comprises an opening 45.

In some embodiments, the device 100 may comprise one or more clips 51, 61, 71, 81, which may be coupled to the holder 31, directly or indirectly, and which may be suitable for holding or supporting lines, wires, and conduits that may be relatively smaller in size or number than lines, wires, and conduits that the holder 31 may be suitable for holding or supporting. Preferably, each clip 51, 61, 71, 81, may have a clip base 52, 62, 72, 82 and a clip arm 53, 63, 73, 83. A clip base 52, 62, 72, 82, may couple the clip 51, 61, 71, 81, to the holder 31 or to another clip 51, 61, 71, 81, and the clip arm 53, 63, 73, 83, of the clip 51, 61, 71, 81, may be coupled to its clip base 52, 62, 72, 82.

In some embodiments, the device 100 may include a first clip 51 that may be coupled to the holder 31, and the first clip 51 may have a first clip base 52, and a first clip arm 53. The first clip base 52 may be coupled to the first holder arm 33, and a first clip channel 54 may be formed between the first holder arm 33, the first clip base 52, and the first clip arm 53. In some embodiments, the device 100 may include a second clip 61 that may be coupled to the holder 31, and the second clip 61 may have a second clip base 62, and a second clip arm 63. The second clip base 62 may be coupled to the second holder arm 34, and a second clip channel 64 may be formed between the second holder arm 34, the second clip base 62, and the second clip arm 63. In some embodiments, the device 100 may include a third clip 71 that may be coupled to the first clip 51, and the third clip 71 may have a third clip base 72, and a third clip arm 73. The third clip base 72 may be coupled to the first clip arm 53, and a third clip channel 74 may be formed between the first clip arm 53, the third clip base 62, and the third clip arm 63. In some embodiments, the device 100 may include a fourth clip 81 that may be coupled to the second clip 61, and the fourth clip 81 may have a fourth clip base 82, and a fourth clip arm 83. The fourth clip base 82 may be coupled to the second clip arm 83, and a fourth clip channel 84 may be formed between the second clip arm 83, the fourth clip base 82, and the fourth clip arm 83.

In preferred embodiments, one or more clips 51, 61, 71, 81, may each comprise a clip distal end 55, 65, 75, 85, that may be flared away from the holder arm 33, 34 or clip 51, 61, 71, 81, that each clip 51, 61, 71, 81, is coupled to. Clip distal ends 55, 65, 75, 85, may comprise the end of a clip arm 53, 63, 73, 83, that is farthest from its respective clip base 52, 62, 72, 82. Generally, clip distal ends 55, 65, 75, 85, that are flared away from the object that the clip 51, 61, 71, 81, is coupled to may facilitate the entry of lines, wires, and conduits into its clip channel 54, 64, 74, 84.

In some embodiments, the holder 31 may comprise one or more holder protrusions 38, 39, that may be coupled to a holder arm 33, 34. A holder protrusion 38, 39, may comprise a ridge, bump, or other protrusion of any size and shape that may extend away from a holder arm 33, 34, and towards a clip 51, 61, 71, 81, and that may be positioned in or proximate to a clip channel 54, 64, 74, 84. Generally, a holder protrusion 38, 39, may help to maintain a line, wire, conduit, etc., in a clip channel 54, 64, by functioning to narrow a portion of the clip channel 54, 64, so that a line, wire, conduit, etc., must be pressed past the holder protrusion 38, 39, in order to move the line, wire, conduit, etc., into and out of the clip channel 54, 64. In a similar manner, a clip distal end 51, 61, of a clip 51, 61, may be flared away from the holder arm 33, 34, that the clip 51, 61, is coupled to, and therefore flared into the clip channel 74, 84, of the adjacent clip 71, 81, so that the clip distal end 51, 61, may function to narrow a portion of the adjacent clip channel 74, 84, so that a line, wire, conduit, etc., must be pressed past the clip distal end 51, 61, in order to move the line, wire, conduit, etc., into and out of the adjacent clip channel 74, 84.

In preferred embodiments, the holder 31 may comprise a first holder protrusion 38 that may be coupled to the first holder arm 33, and the first holder protrusion 38 may be positioned within the first clip channel 54. In further preferred embodiments, the holder 31 may comprise a second holder protrusion 39 that may be coupled to the second holder arm 34, and the second holder protrusion 39 may be positioned within the second clip channel 64.

While some exemplary shapes and sizes have been provided for elements of the device 100, it should be understood to one of ordinary skill in the art that the retainer 11, holder 31, clips 51, 61, 71, 81, and any other element described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Additionally, while some materials have been provided, in other embodiments, the elements that comprise the device 100 may be made from or may comprise durable materials such as aluminum, steel, other metals and metal alloys, wood, hard rubbers, hard plastics, fiber reinforced plastics, carbon fiber, fiberglass, resins, polymers or any other suitable materials including combinations of materials. Additionally, one or more elements may be made from or may comprise durable and slightly flexible materials such as soft plastics, silicone, soft rubbers, or any other suitable materials including combinations of materials. In some embodiments, one or more of the elements that comprise the device 100 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 100 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 100 may be coupled by being one of connected to and integrally formed with another element of the device 100.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A conduit attachment device, the device comprising:
a retainer having a retainer base, a first retainer arm, a second retainer arm, a retainer strap, and a fastener, wherein the first retainer arm and the second retainer arm are coupled to the retainer base, wherein a retainer channel is formed between the first retainer arm, the retainer base, and the second retainer arm, wherein the retainer strap is coupled to the first retainer arm and the fastener is coupled to the second retainer arm, wherein the fastener is configured to be coupled to the retainer strap;
a holder coupled to the retainer, the holder having a holder base, a first holder arm, and a second holder arm, wherein the first holder arm and the second holder arm are coupled to the holder base, and wherein a holder channel is formed between the first holder arm, the holder base, and the second holder arm; and
a first clip coupled to the holder, the first clip having a first clip base, and a first clip arm, wherein the first clip base is coupled to the first holder arm, and wherein a first clip channel is formed between the first holder arm, the first clip base, and the first clip arm.

2. The device of claim 1, wherein the first retainer arm is elongated in shape and the second retainer arm is elongated in shape, and wherein the first retainer arm extends away from the retainer base in a direction that is substantially parallel to a direction that the second retainer arm extends away from the retainer base.

3. The device of claim 1, wherein the first holder arm is elongated in shape and the second holder arm is elongated in shape, and wherein the first holder arm extends away from the holder base in a direction that is substantially parallel to a direction that the second holder arm extends away from the holder base.

4. The device of claim 1, wherein the fastener is configured to be removably coupled to the retainer strap.

5. The device of claim 1, wherein the holder and the retainer are rotatably coupled together via a movable coupling.

6. The device of claim 5, wherein the movable coupling comprises a male member and a female member, and wherein the male member is rotatably coupled within the female member.

7. The device of claim 1, wherein the first retainer arm comprises a first retainer barb, and wherein the second retainer arm comprises a second retainer barb.

8. The device of claim 1, wherein the holder comprises a holder protrusion that is coupled to the first holder arm, and wherein the holder protrusion is positioned within the first clip channel.

9. The device of claim 1, wherein the first holder arm comprises a first holder distal end and the second holder arm comprises a second holder distal end, and wherein the first holder distal end and the second holder distal end are flared away from each other.

10. The device of claim 1, wherein the first clip comprises a first clip distal end, and wherein the first clip distal end is flared away from the first holder arm.

11. A conduit attachment device, the device comprising:
a retainer having a retainer base, a first retainer arm, a second retainer arm, a retainer strap, and a fastener, wherein the first retainer arm and the second retainer arm are coupled to the retainer base, wherein a retainer channel is formed between the first retainer arm, the retainer base, and the second retainer arm, wherein the retainer strap is coupled to the first retainer arm and the fastener is coupled to the second retainer arm, wherein the retainer strap has a fastener aperture, and wherein the fastener is configured to be coupled to the retainer strap by inserting the fastener into the fastener aperture;
a holder coupled to the retainer, the holder having a holder base, a first holder arm, and a second holder arm, wherein the first holder arm and the second holder arm are coupled to the holder base, and wherein a holder channel is formed between the first holder arm, the holder base, and the second holder arm;
a first clip coupled to the holder, the first clip having a first clip base, and a first clip arm, wherein the first clip base is coupled to the first holder arm, and wherein a first clip channel is formed between the first holder arm, the first clip base, and the first clip arm; and
a second clip coupled to the holder, the second clip having a second clip base, and a second clip arm, wherein the second clip base is coupled to the second holder arm, and wherein a second clip channel is formed between the second holder arm, the second clip base, and the second clip arm.

12. The device of claim 11, wherein the first retainer arm is elongated in shape and the second retainer arm is elongated in shape, and wherein the first retainer arm extends away from the retainer base in a direction that is substantially parallel to a direction that the second retainer arm extends away from the retainer base.

13. The device of claim 11, wherein the first holder arm is elongated in shape and the second holder arm is elongated in shape, and wherein the first holder arm extends away from the holder base in a direction that is substantially parallel to a direction that the second holder arm extends away from the holder base.

14. The device of claim 11, wherein the fastener is configured to be removably coupled to the retainer strap.

15. The device of claim 11, wherein the holder and the retainer are rotatably coupled together via a movable coupling.

16. The device of claim 15, wherein the movable coupling comprises a male member and a female member, and wherein the male member is rotatably coupled within the female member.

17. The device of claim 11, wherein the first retainer arm comprises a first retainer barb and the second retainer arm comprises a second retainer barb, and wherein the first retainer barb and the second retainer barb extend towards each other.

18. The device of claim 11, wherein the holder comprises a first holder protrusion that is coupled to the first holder arm, wherein the first holder protrusion is positioned within the first clip channel, wherein the holder comprises a second holder protrusion that is coupled to the second holder arm, wherein the second holder protrusion is positioned within the second clip channel.

19. The device of claim 11, wherein the first holder arm comprises a first holder distal end and the second holder arm comprises a second holder distal end, and wherein the first holder distal end and the second holder distal end are flared away from each other.

20. The device of claim 11, wherein the first clip comprises a first clip distal end, and wherein the first clip distal end is flared away from the first holder arm, wherein the second clip comprises a second clip distal end, and wherein the second clip distal end is flared away from the second holder arm.
